(19) United States
(12) Patent Application Publication
(10) Pub. No.: US 2022/0387647 A1
(43) Pub. Date: Dec. 8, 2022

(54) Self-sanitizing Poles

(71) Applicant: Mirza Faizan, Irving, TX (US)

(72) Inventors: Mirza Faizan, Irving, TX (US); Mirza Rizwan, Patna (IN); Raghavi Danduboyina, Southlake, TX (US); Shayaan Jafri, Southlake, TX (US); Sarah Khurshid, Irving, TX (US); Maya Kusumakar, Grapevine, TX (US); Vesaam Malik, Westlake, TX (US); Nadia Mustafa, Southlake, TX (US); Nauman Mustafa, Southlake, TX (US); Azka Rafique, Southlake, TX (US); Sarrinah Rafique, Southlake, TX (US); Abdurraheem Sheikh, Irving, TX (US); Humza Sheikh, Southlake, TX (US)

(21) Appl. No.: 17/339,483

(22) Filed: Jun. 4, 2021

(51) Int. Cl. A61L 2/24; E05B 1/00; G01S 17/04; A61L 2/10

(52) U.S. Cl. CPC A61L 2/24; E05B 1/0069; G01S 17/04; A61L 2/10; A61L 2202/14; A61L 2202/11

(57) ABSTRACT

The present invention relates to a self-sanitizing support handle and a method of operating the same. A self-sanitizing support handle is disclosed. The support handle comprising: a rotating mechanism; one or more sensors to determine a proximity of user; and a plurality of lights on said handle, wherein one or more lights from said plurality of lights gets activated upon determination of absence of said user in proximity based on said sensor output and upon activation sanitizes an area of said support handle where said absence of said user is determined.

### TECHNICAL FIELD

The invention relates to a self-sanitizing pole. More specifically, the invention relates to ultraviolet light-based self-sanitizing pole. Furthermore specifically, the invention relates to a self-powered ultraviolet light-based self-sanitizing pole, especially in trains.

### BACKGROUND OF THE INVENTION

In general, it has been noted that every person is exposed to enormous microbes on a daily basis. These microbes may include viral and bacterial diseases and contribute to spreading communicable diseases. Diseases may be transmitted by direct contact when an infected person touches another person or by indirect contact when there is no direct person-to-person contact. Most often, indirect transfer of disease occurs when an infected person touches a surface and leaves behind microbes on the surface. An uninfected person subsequently touches the same surface and the microbes adhere to the skin.

However, the available preventative measures are impractical to use every time a potentially contaminated surface is touched. Furthermore, poles in public transportation are constantly being touched by many people; therefore they contain a lot of germs that are not being cleaned before a new person uses the poles. The lack of sanitization between each use can lead to the spread of germs, infections, viruses, etc. The expected outcome should be an automatic way to disinfect, sanitize, and sterilize the poles between each use efficiently to get rid of the spread of germs, infections, viruses, etc w/out disturbing passengers.

The presently available other alternates of sanitization does not provide an effective solution to the issue of sanitization. Therefore, there is a need for efficiently and effectively sanitizing the poles in public transportation.

### SUMMARY OF THE INVENTION

An embodiment is directed to self-sanitizing support handle, said support handle comprising: a rotating mechanism; one or more sensors to determine a proximity of user; and a plurality of lights on said handle, wherein one or more lights from said plurality of lights gets activated upon determination of absence of said user in proximity based on said sensor output and upon activation sanitizes an area of said support handle where said absence of said user is determined.

In addition to one or more of the features described above or below, or as an alternative, the support handle further comprising a power unit.

In addition to one or more of the features described above or below, or as an alternative, wherein the sanitization is activated for a pre-determined time interval.

In addition to one or more of the features described above or below, or as an alternative, the support handle comprising a light indication unit configured to reflect a status of the plurality of lights.

In addition to one or more of the features described above or below, or as an alternative, the support handle further comprising an outer covering of plastic, said plastic covering activates upon determination of absence of any user in proximity.

In addition to one or more of the features described above or below, or as an alternative, the support handle comprising one or more sensors configured to detect presence of one or more users.

In addition to one or more of the features described above or below, or as an alternative, the support handle comprising one or more sensors configured to detect presence of one or more users.

In addition to one or more of the features described above or below, or as an alternative, the support handle comprising a control circuit for causing the UV-C disinfectant to be conveyed to the handle in intervals, wherein the intervals are responsive to an external condition and the control circuit if free of any manual activation.

An embodiment is directed to method of operating the self-sanitizing support handle.

An exemplary embodiment may be directed to an ultraviolet light-based self-sanitizing support handle. The self-sanitizing support handle comprises a thermal detector or a sensor that may sense a presence of human being. The UV emission assembly triggers when the sensors determine that no user is in close proximity of the support handle.

### OBJECTS OF THE INVENTION

An object of the disclosed invention is to provide a self-sanitizing support handle, basically of pole of the vehicles.

Yet another object of the disclosed invention is to provide ultraviolet light-based support handle.

Yet another objective of the invention is to provide an ultraviolet light-based support handle that detects human presence.

Yet another objective of the invention is to provide a self-powered ultraviolet light-based support handle to kill germs and viruses from the support handle.

Yet another objective of the invention is to provide a self-powered sensor based automatic ultraviolet light-based support handle to kill germs and viruses from the support handle.

Yet another objective of the invention is to provide a self-powered ultraviolet light-based support handle to kill germs and viruses from the support handle in a pre-determined time interval.

Yet another objective of the invention is to provide a support handle sanitization technique which selectively sanitizes the probable affected area.

### BRIEF DESCRIPTION OF THE DRAWINGS

The accompanying drawings illustrate various embodiments of systems, methods, and other aspects of the disclosure. Any person having ordinary skill in the art will appreciate that the illustrated element boundaries (e.g., boxes, groups of boxes, or other shapes) in the figures represent one example of the boundaries. It may be that in some examples, one element may be designed as multiple elements or that multiple elements may be designed as one element. In some examples, an element shown as an internal component of one element may be implemented as an external component in another, and vice versa. Furthermore, elements may not be drawn to scale.

Various embodiments will hereinafter be described in accordance with the appended drawings, which are provided to illustrate, and not to limit the scope in any manner, wherein like designations denote similar elements, and in which:

FIG. 1 is a self-sanitizing support handle in which various embodiments may be implemented;

FIG. 2 is a covering mechanism of said support handle for complete sanitization;

FIG. 3 illustrates an exemplary self-sanitizing support handle.

### DETAILED DESCRIPTION OF DRAWINGS

The present disclosure is best understood with reference to the detailed figures and description set forth herein. Various embodiments are discussed below with reference to the figures. However, those skilled in the art will readily appreciate that the detailed descriptions given herein with respect to the figures are simply for explanatory purposes as the methods and systems may extend beyond the described embodiments. For example, the teachings presented and the needs of a particular application may yield multiple alternate and suitable approaches to implement the functionality of any detail described herein. Therefore, any approach may extend beyond the particular implementation choices in the following embodiments described and shown.

References to “one embodiment,” “an embodiment,” “at least one embodiment,” “one example,” “an example,” “for example,” and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment.

It is noted that various connections are set forth between elements in the following description and in the drawings (the contents of which are included in this disclosure by way of reference). It is noted that these connections in general and, unless specified otherwise, may be direct or indirect and that this specification is not intended to be limiting in this respect. In this respect, a coupling between entities may refer to either a direct or an indirect connection.

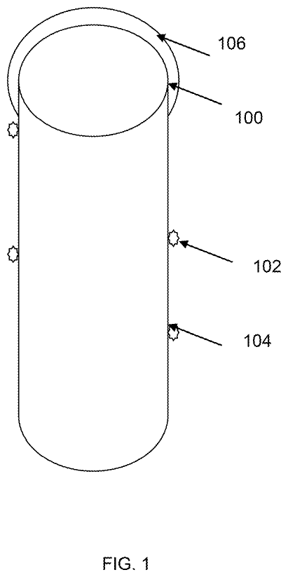

FIG. 1 is a self-sanitizing support handle in a closed environment in which various embodiments may be implemented.

FIG. 1 shows self-sanitizing support handle 100 that includes one or more sensors 102, an ultra violet rays light 104, and a cover 106. Various devices in the system environment 100 may be interconnected. The self-sanitizing support handle 100 is closed in this instance. Hence, the UV source is in closed circuit and may operate in pre-determined interval, after the first instance of operation.

The pole or the support handle 100 may be made up of multiple components and materials thereof. The pole 100 may be hollow from inside so that UVC LED lights are inserted Furthermore, the pole 100 may be made in such a manner that the UV-C lights 104 present inside the pole or support handle 100 may be enabled to emit the UV-C to the outer layer of the support handle and thereby kills one or more germs/viruses present on the support handle. Furthermore, as soon as the user removes the hand or support from the support handle, the support handle gets intimation that the person has left, and thereby the UV-C lights of that specific place gets activated and sanitizes the place.

The UV-C lights 104 may correspond to ultra violet emitting lights. The UV-C lights may be self powered lights that may be empowered by one or more sources of power as solar based energy generation. The number of lights inside the support handle may be based on the length and the user requirement. The user may alter the number of lights in functional state as per the frequency of usage. In an embodiment, an internal protection circuit may be placed along with the UV-C lights, that may protect the lights and moreover from any power issues.

Further, the support handle may be made up of aluminium or a wooden based material in which the UV-C lights are placed. The placement of the UV-C lights in the support handle may be based as per the need of the user. The user requirement may be based on frequency of usage of the support handle or strict sanitization requirement for a longer time.

The cover 106 of the support handle may be made up of any material to cover the overall door handle assembly. In an embodiment, the cover 106 may be pulled up from a downward direction for covering the support handle, and thereafter the entire sanitization process may take place.

In an embodiment of the present invention, the support handle and the cover may be water proof covers. Once the users are absent from the close proximity, then the entire support handle may be washed deeply by one or more other sources of sanitization, as soapy water. The soapy water may be present in the over head tanks, and may be dissipated from one or more pores available on the support handle.

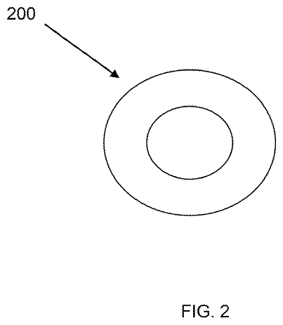

FIG. 2 illustrates a covering mechanism of the support handle for complete sanitization.

As shown in FIG. 200, the support handle may have an outer cover. This outer cover may only get lifted up at an instance, when the sanitization process has to take place on the entire support handle. The outer cover may be hydraulically enabled cover.

In an embodiment of the present invention, the hydraulic mechanism of the outer cover gets activated as it receives signals from the processor associated with the support handle. The processor may be communicatively attached to one or more sensors. The one or more sensors may be configured to determine the presence of the users in close proximity of the support handle. As soon as the sensors detect that there is no user present in proximity, then at that instance processor acts and trigger the hydraulic function of the outer cover. Thus, the outer cover may lifts up and covers the support handle. Upon complete cover, the UV-C system gets activated and the entire support handle gets sanitized.

In an embodiment of the present invention, time period of the sanitization may be based on a pre-determined time interval for killing germs and viruses that may have arrived from the users onto the support handle.

In an embodiment of the present invention, the UV-C lights may be placed upto four sides of the handle. In an embodiment, the number of lights may be determined at initial installation state based on the requirements and dimensions of the support handle.

Furthermore, in an embodiment, the support handle may comprise a light-based alarming feature and the light may be operative in multiple modes as Green, Red or Yellow. The green light shows that the support handle is sanitized and is ready to use. The yellow may indicate that the support handle is recently used by a user and is getting prepared to be reused. Furthermore, the red light shows that the support handle is not sanitized and the user should wait for the support handle to get sanitize before the use.

Additionally, in an embodiment of the present invention, the Self-sanitizing support handle may comprise an adjustable interval trigger mechanism. The adjustable interval system may be configured to operate the auto sanitization of the support handle after a pre-determined time intervals. The time interval may be a user defined time interval as per the frequency of the use of support handle.

The adjustable intervals may be configured in an automatic manner or may be a user driven. The automatic time intervals may be pre-determined time intervals that may be adjusted by the controller. The controller may be communicatively coupled with the support handle.

In an embodiment of the present invention, the self-sanitizing support handle may comprise a remotely connected controller. The controller may include a microprocessor (not shown) and associated memory. The memory may store operational parameters of the unit, as well as software to control operation of the door. The microprocessor may perform operations according to the software using the operational parameters as well as other parameters or information obtained by the controller.

In an embodiment of the present invention, the support handle may also have communicatively attached display device. The display device may display the infected are or the used area by the user. This may alarm the other users not to touch the infected area, prior to sanitization.

In an embodiment of the present invention, a method may be disclosed. The method may be configured to enable the function of the sanitization in the support handle. The entire sanitization process may be processor driven and may be enabled by a protection circuit. The protection circuit may enable the function to perform sanitization in a manual manner.

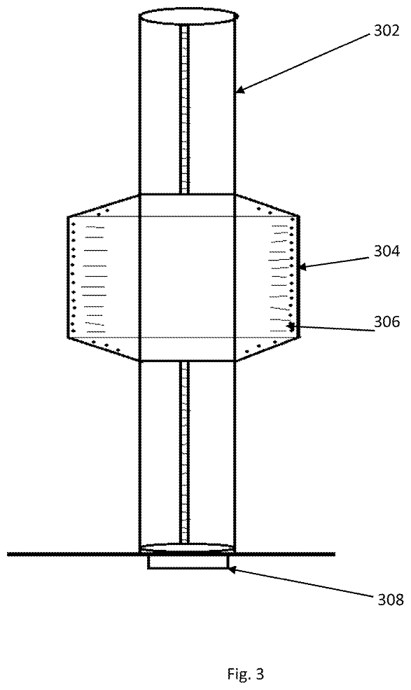

FIG. 3 discloses an exemplary self-sanitizing support handle. The support handle 302 may be mechanically attached to the roof of the bus in upward direction and by the floor of the bus in downward direction. The support handle 302 may be equipped with a disinfectant ring 304. The disinfectant ring 304 may comprise plurality of UVC LED lights 306. The UVC lights may be enabled upon detection that nobody is present nearby the support handle and henceforth disinfectant mechanism gets activated and produces the desired results. There is also a charging station 308 electronically attached with the self-sanitizing support handle, which provides desired power requirements to enable the system.

In an exemplary embodiment, a group of 20 people boards the vehicle, say a bus. The bus may have 4 poles at equal distances. Out of 20, 10 peoples holds the support handle. Now, 5 people leave the bus at first stop. As soon as they leave and the processor determines that nobody is in close proximity of poles, then the system may sanitize that particular area. At the same time, the exposed area may also be highlighted and may also be displayed in the display unit. The remaining 5 people still holds the support handle, and thereby in such places the sanitization system may not be activated. Now at second stop, all of the people from the bus gets down. At this instance the entire system may get activated and the cover may be laid over the pole and sanitization may take place by one or more disclosed methods of sanitization.

Various embodiments of the self-sanitizing support handle have been disclosed. However, it should be apparent to those skilled in the art that modifications in addition to those described, are possible without departing from the inventive concepts herein. The embodiments, therefore, are not restrictive, except in the spirit of the disclosure. Moreover, in interpreting the disclosure, all terms should be understood in the broadest possible manner consistent with the context. In particular, the terms “comprises” and “comprising” should be interpreted as referring to elements, components, or steps, in a non-exclusive manner, indicating that the referenced elements, components, or steps may be present, or utilized, or combined with other elements, components, or steps that are not expressly referenced.

It is noted that various connections are set forth between elements in the description and in the drawings (the contents of which are included in this disclosure by way of reference). It is noted that these connections in general and, unless specified otherwise, may be direct or indirect and that this specification is not intended to be limiting in this respect. In this respect, a coupling between entities may refer to either a direct or an indirect connection.

The disclosed methods and systems, as illustrated in the ongoing description or any of its components, may be embodied in the form of a computer system. Typical examples of a computer system include a general-purpose computer, a programmed microprocessor, a micro-controller, a peripheral integrated circuit element, and other devices, or arrangements of devices that are capable of implementing the steps that constitute the method of the disclosure.

The computer system comprises a computer, an input device, a display unit and the Internet. The computer further comprises a microprocessor. The microprocessor is connected to a communication bus. The computer also includes a memory. The memory may be Random Access Memory (RAM) or Read Only Memory (ROM). The computer system further comprises a storage device, which may be a hard-disk drive or a removable storage drive, such as, a floppy-disk drive, optical-disk drive, and the like. The storage device may also be a means for loading computer programs or other instructions into the computer system. The computer system also includes a communication unit. The communication unit allows the computer to connect to other databases and the Internet through an input/output (I/O) interface, allowing the transfer as well as reception of data from other sources. The communication unit may include a modem, an Ethernet card, or other similar devices, which enable the computer system to connect to databases and networks, such as, LAN, MAN, WAN, and the Internet. The computer system facilitates input from a user through input devices accessible to the system through an I/O interface.

In order to process input data, the computer system executes a set of instructions that are stored in one or more storage elements. The storage elements may also hold data or other information, as desired. The storage element may be in the form of an information source or a physical memory element present in the processing machine.

The programmable or computer-readable instructions may include various commands that instruct the processing machine to perform specific tasks, such as steps that constitute the method of the disclosure. The systems and methods described can also be implemented using only software programming or using only hardware or by a varying combination of the two techniques. The disclosure is independent of the programming language and the operating system used in the computers. The instructions for the disclosure can be written in all programming languages including, but not limited to, “C,” “C++,” “Visual C++,” Java, and “Visual Basic.” Further, the software may be in the form of a collection of separate programs, a program module containing a larger program or a portion of a program module, as discussed in the ongoing description. The software may also include modular programming in the form of object-oriented programming. The processing of input data by the processing machine may be in response to user commands, the results of previous processing, or from a request made by another processing machine. The disclosure can also be implemented in various operating systems and platforms including, but not limited to, “Unix,” “DOS,” “Android,” “Symbian,” and “Linux.”

The programmable instructions can be stored and transmitted on a computer-readable medium. The disclosure can also be embodied in a computer program product comprising a computer-readable medium, or with any product capable of implementing the above methods and systems, or the numerous possible variations thereof.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

A person having ordinary skills in the art will appreciate that the system, modules, and sub-modules have been illustrated and explained to serve as examples and should not be considered limiting in any manner. It will be further appreciated that the variants of the above disclosed system elements, or modules and other features and functions, or alternatives thereof, may be combined to create other different systems or applications.

Although a few implementations have been described in detail above, other modifications are possible. Moreover, other mechanisms for performing the systems and methods described in this document may be used. In addition, the logic flows depicted in the figures may not require the particular order shown, or sequential order, to achieve desirable results. Other steps may be provided, or steps may be eliminated, from the described flows, and other components may be added to, or removed from, the described systems. Accordingly, other implementations are within the scope of the following claims.

What is claimed is:

1. A self-sanitizing support handle, said support handle comprising:
a rotating mechanism;
one or more sensors to determine a proximity of user; and
a plurality of lights on said handle, wherein one or more lights from said plurality of lights gets activated upon determination of absence of said user in proximity based on said sensor output and upon activation sanitizes an area of said support handle where said absence of said user is determined.

2. The self-sanitizing support handle as claimed in claim 1 further comprising a power unit.

3. The self-sanitizing support handle as claimed in claim 1, wherein said sanitization is activated for a pre-determined time interval.

4. The self-sanitizing support handle as claimed in claim 1, further comprising a light indication unit configured to reflect a status of the plurality of lights.

5. The self-sanitizing support handle as claimed in claim 1 further comprising an outer covering of plastic, said plastic covering activates upon determination of absence of any user in proximity.

6. The self-sanitizing support handle as claimed in claim 1 further comprising one or more sensors configured to detect presence of one or more users.

7. The self-sanitizing support handle as claimed in claim 1 further comprising a control circuit for causing the UV-C disinfectant to be conveyed to the handle in intervals, wherein the intervals are responsive to an external condition and the control circuit if free of any manual activation.

8. The self-sanitizing support handle as claimed in claim 1 further comprising a display module configured to display the infected portion of said support handle.

9. A method of operating, said self-sanitizing support handle as claimed in claims 1 to 8.
